(19) United States
(12) Patent Application Publication
(10) Pub. No.: US 2018/0214060 A1
(43) Pub. Date: Aug. 2, 2018

(54) Neurological Brain Disorder Treatment

(71) Applicant: Victoria J. Brown, Tustin, CA (US)

(72) Inventor: Victoria J. Brown, Tustin, CA (US)

(21) Appl. No.: 15/879,905

(22) Filed: Jan. 25, 2018

Related U.S. Application Data
(60) Provisional application No. 62/459,560, filed on Feb. 15, 2017.
(60) Provisional application No. 62/453,987, filed on Feb. 2, 2017.

(51) Int. Cl. A61B 5/16

(52) U.S. Cl. CPC A61B 5/163; A61B 5/168

(57) ABSTRACT

A method of treating neurological disorders is provided using an EMDR device having at least two vibrating panels, where the method includes attaching a first vibrating panel to a first side of a patient's body, attaching a second vibrating panel to a second side of a patient's body, and then activating the EMDR controller to activate the first and second vibrating panels to create bilateral brain stimulation, resulting in more uniform brain waves.

### RELATED APPLICATION

This application is a non-provisional application claiming priority to provisional application U.S. Ser. No. 62/453,987, filed Feb. 2, 2017, and provisional application U.S. Ser. No. 62/459,560, filed Feb. 15, 2017, the entire contents of both of which are incorporated herein by reference.

### BACKGROUND

The present invention relates to a method of using an Eye Movement Desensitization And Reprocessing (EMDR) device with vibrating panels to treat attention deficit disorder (ADD) and other neurological disorders by causing the brain to experience bilateral stimuli.

Currently EMDR devices are used to treat patients with Post Traumatic Stress Disorder (PTSD). The device is typically equipped with a controller and two paddles, or panels, with one held in each hand. When the controller is activated, the panels are vibrated to help move the PTSD patients from experiencing the trauma in the traumatized right hemisphere to experiencing the event in both hemispheres.

Children and adults with ADD do not have neuro-traditional brains, so they are constantly searching for additional stimuli to enable connectivity in the brain. Current treatments often involve amphetamines to fulfill their need for additional stimulation and assist their neuro-non-traditional brains to function daily.

### DETAILED DESCRIPTION OF CERTAIN EMBODIMENTS

During treatment with attention deficit disorder patients, I have found that inversion of the body or blood flow and/or bilateral movement can balance the brain immediately and increase length of functioning. By attaching the panels of an EMDR device to the left and right sides of the body (e.g. shoulder blades), an ADD brain immediately connects with the stimuli and fills the need to be distracted, allowing for substantially greater focus. It is healthy for the brain to desire bilateral movement, which is why swimming or rocking a baby is helpful, and also why autistic children rock themselves. These actions naturally sooth the brain decreasing pressure in the frontal lobe.

I have tested this theory specifically with a NeuroTek™ Audio Scan EMDR device and IASIS™ MCN (micro current neurofeedback) technology. Results in balanced brainwaves have been measured by testing ADD and PTSD patients with neurological software, such as IASIS, which treats imbalanced and erratic brainwaves and graphs the changing of brainwaves over a period of time. Patients were tested using IASIS first without embodiments of the invention attached to the patient's body, then again with the panels of the EMDR device attached, for example, to the patient's shoulder blades (a first adhesive panel to the first side of the body and a second adhesive panel to the second side of the body). Prior to applying adhesive panels to the patient's body, the brain waves are observed to be erratic and unbalanced (a consistent neuro nontraditional brain wave state). Once the panels of the EMDR device are applied, the controller can be activated and then the patient's brain waves immediately change to an even and uniform pace, again as measured on IASIS. Attaching the panels to a patient's body allows the patient to proceed through the day normally without having to hold anything in one's hand. The attached panels permit the system to be hidden under the patient's clothing, avoiding any embarrassment associated with employing the device.

I have additionally tested this theory specifically with a NeuroTek™ Audio Scan EMDR device and TOVA diagnostic software, which measures the key components of attention and self-control, variability (consistency), response time (speed), commissions (impulsivity), and omissions (focus and vigilance), It is contemplated that monitoring of the patient at any time may be performed using an electroencephalogram, for example, to determine whether someone is in an agitated state and/or to monitor the theta brain waves.

It is also contemplated that adhesive panels and EMDR controller could be used for a child to wear while in class to increase focus, while decreasing disturbances in the classroom. The plan would be to fulfill the need of a natural treatment, which would work instantly or assist the current pharmaceutical medications.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A method of treating neurological disorders using an EMDR device having at least two vibrating panels, the method comprising (a) attaching a first vibrating panel to a first side of a patient's body, (b) attaching a second vibrating panel to a second side of a patient's body, and (c) activating the EMDR controller to activate both first and second vibrating panels to create bilateral brain stimulation, resulting in more uniform brain waves.

2. The method of claim 1, where attaching comprises adhesively attaching.

3. The method of claim 1, where the patient's body comprises the patient's shoulder blades.
